(19) United States
(12) Patent Application Publication
(10) Pub. No.: US 2026/0294780 A1
(43) Pub. Date: Oct. 1, 2026

(54) COMPOSITION COMPRISING IONICALLY CROSSLINKED HIGH MOLECULAR WEIGHT CHITOSAN

(71) Applicant: L'OREAL, Paris (FR)

(72) Inventors: Mariko YAMAMOTO, Kawasaki-shi (JP); Takehiko KASAI, Kawasaki-shi (JP); Tatsushi ISOJIMA, Kawasaki-shi (JP)

(21) Appl. No.: 19/476,105

(22) PCT Filed: Apr. 5, 2024
(86) PCT No.: PCT/JP2024/080051

§ 371 Date: Oct. 16, 2025

(30) Foreign Application Priority Data
Apr. 18, 2023 (JP) 2023-067933
May 24, 2023 (FR) 2305128

(51) Int. Cl. A61K 8/73; A61K 8/14; A61K 8/36; A61Q 5/06

(52) U.S. Cl. CPC A61K 8/736; A61K 8/14; A61K 8/361; A61Q 5/06

(57) ABSTRACT

The present invention relates to a composition comprising: (a) at least one cationic polymer; (b) at least one monovalent non-polymeric acid or a salt thereof, and (c) at least one non-polymeric acid having two or more pKa values or a salt thereof, wherein the (a) cationic polymer is selected from chitosans with a molecular weight of more than 20,000. The composition according to the present invention can provide keratin fibers such as hair with good alignment and shape recovery effects, and can include at least one environmentally-friendly ingredient.

### TECHNICAL FIELD

The present invention relates to a composition including an ionically crosslinked high molecular weight chitosan, as well as a cosmetic process using the composition.

### BACKGROUND ART

The formulation of environmentally-friendly cosmetic products, which are designed and developed considering environmental issues, is becoming a major goal in an effort to meet global challenges.

It is therefore essential to propose more sustainable compositions, preparation processes and ingredients to address these environmental concerns.

In this context, it is important to develop new cosmetic compositions with a better carbon footprint, particularly by promoting the use of renewable raw materials and/or materials with a good index of naturalness and/or materials of natural origin and, more particularly, materials of plant origin while reducing the use of compounds of petrochemical origin.

As is known in the art, certain cosmetic composition and any other compositions use a polyion complex, which is formed with an anionic polymer and a cationic polymer.

For example, WO 2021/171909 discloses a composition which comprises at least one polyion complex particle comprising at least one cationic polymer, at least one anionic polymer and at least one non-polymeric acid having two or more pKa values.

### DISCLOSURE OF INVENTION

There is a need for a composition which can provide keratin fibers such as hair with good alignment and shape recovery effects while including environmentally-friendly ingredient(s).

The alignment effect means that keratin fibers can be easily arranged in a desired line, such as a straight line. The shape recovery effect means that keratin fibers can easily return to their original shape.

Thus, a first objective of the present invention is to provide a composition which can provide keratin fibers such as hair with good alignment and shape recovery effects.

In addition, a second objective of the present invention is to provide a composition which can include at least one environmentally-friendly ingredient.

The above objectives of the present invention can be achieved by a composition, preferably a cosmetic composition, and more preferably a cosmetic composition for keratin fibers such as hair, comprising:
(a) at least one cationic polymer;
(b) at least one monovalent non-polymeric acid or a salt thereof, and
(c) at least one non-polymeric acid having two or more pKa values or a salt thereof, wherein
the (a) cationic polymer is selected from chitosans with a molecular weight (Da) of more than 20,000.

The amount of the (a) cationic polymer(s) in the composition according to the present invention may be from 0.01% to 15% by weight, preferably from 0.05% to 10% by weight, and more preferably from 0.1% to 5% by weight, relative to the total weight of the composition.

The (b) monovalent non-polymeric acid or a salt thereof may be a monovalent non-polymeric organic acid or a salt thereof, preferably a monovalent carboxylic acid or a salt thereof, and more preferably lactic acid or a salt thereof.

The amount of the (b) monovalent non-polymeric acid(s) or salt(s) thereof in the composition according to the present invention may be from 0.01% to 15% by weight, preferably from 0.05% to 10% by weight, and more preferably from 0.1% to 5% by weight, relative to the total weight of the composition.

The (c) non-polymeric acid having two or more pKa values or a salt thereof may be an organic acid or a salt thereof, preferably a hydrophilic or water-soluble organic acid with at least one phosphoric acid group and/or at least two carboxylic acid groups, or salts thereof, and more preferably phosphoric acid, diphosphoric acid, citric acid, tartaric acid, or salts thereof.

The amount of the (c) non-polymeric acid(s) having two or more pKa values or salt(s) thereof in the composition according to the present invention may be from 0.001% to 10% by weight, preferably from 0.003% to 5% by weight, and more preferably from 0.005% to 1% by weight, relative to the total weight of the composition.

Preferably, the composition according to the present invention comprises (d) water.

The amount of the (d) water in the composition according to the present invention may be from 50% to 99% by weight, preferably from 60% to 97% by weight, and more preferably from 70% to 95% by weight, relative to the total weight of the composition.

The composition according to the present invention may further comprise (e) at least one oil.

The (e) oil may be selected from plant oils, synthetic ester oils, and mixtures thereof, and preferably from plant oils.

The amount of the (e) oil(s) in the composition according to the present invention may be from 1% to 50% by weight, preferably from 3% to 45% by weight, and more preferably from 5% to 40% by weight, relative to the total weight of the composition.

The composition according to the present invention may further comprise (f) at least one fatty acid.

The (f) fatty acid may be selected from C₄-C₂₆, preferably C₆-C₂₄, more preferably C₈-C₂₂ saturated and unsaturated, linear or branched fatty acids.

The (a) cationic polymer may be hydrophobized by the (f) fatty acid.

The amount of the (f) fatty acid(s) in the composition according to the present invention may be from 0.01% to 15% by weight, preferably from 0.05% to 10% by weight, and more preferably from 0.1% to 5% by weight, relative to the total weight of the composition.

The present invention also relates to a cosmetic process for keratin fibers such as hair, comprising
applying to the keratin fibers the composition according to the present invention, and drying the composition to form a cosmetic film on the keratin fibers.

### BRIEF DESCRIPTION OF DRAWINGS

FIG. 1 shows schematic drawings showing the behaviors of the (a) cationic polymers around a fatty phase, if present, such as an oil droplet, dispersed in an aqueous phase, and the examples of hydrophobicization of the (a) cationic polymer, in one embodiment of the present invention.

FIG. 1A shows a schematic drawing showing the behaviour of the (a) cationic polymers around a fatty phase, if present, such as an oil droplet, dispersed in an aqueous phase, in case of the fatty phase including no (f) fatty acid.

FIG. 1B shows a schematic drawing showing the behaviour of the (a) cationic polymers around a fatty phase, if present, such as an oil droplet, dispersed in an aqueous phase, in case of the fatty phase including (f) fatty acid.

FIG. 1C shows a schematic drawing showing the example of mechanism of hydrophobicization of the (a) cationic polymer by the (f) fatty acid.

### BEST MODE FOR CARRYING OUT THE INVENTION

After diligent research, the inventors have discovered that it is possible to provide a composition which can provide keratin fibers such as hair with good alignment and shape recovery effects, and can include at least one environmentally-friendly ingredient.

Thus, the composition according to the present invention comprises:
(a) at least one cationic polymer;
(b) at least one monovalent non-polymeric acid or a salt thereof, and
(c) at least one non-polymeric acid having two or more pKa values or a salt thereof, wherein
the (a) cationic polymer is selected from chitosans with a molecular weight (Da) of more than 20,000.

The keratin fibers treated with the composition according to the present invention can be easily arranged in a desired line, such as a straight line. Also, the keratin fibers treated with the composition according to the present invention can easily return to their original shape. Therefore, the composition according to the present invention can provide good manageability of keratin fibers such as hair.

The (a) cationic polymer can be ionically crosslinked by the (c) non-polymeric acid having two or more pKa values or a salt thereof. The crosslinked cationic polymer can provide better bonding and shape memory effects between keratin fibers such as hair. Therefore, the crosslinked cationic polymer can provide keratin fibers such as hair with good alignment and shape recovery effects. Thus, the present invention can provide, for example, excellent hair styling effects.

Since chitosans can be obtained from natural resources, the (a) cationic polymer is an environmentally-friendly ingredient. Therefore, the composition according to the present invention can include an environmentally-friendly ingredient.

In addition, the ingredients (b) and (c) can be originated from renewable materials such as plants and/or biodegradable materials. Therefore, the composition according to the present invention can be environmentally-friendly.

Preferably, the composition according to the invention comprises (d) water.

The composition according to the present invention may comprise an aqueous phase comprising the (d) water.

If the composition according to the present invention comprises (e) at least one oil, the composition according to the present invention may comprise fatty phases comprising the (e) oil. If the composition according to the present invention comprises (f) at least one fatty acid, the (f) fatty acid may be present in the fatty phases.

The fatty phases may be dispersed in the aqueous phase. In this case, the fatty phases are discontinuous phases, while the aqueous phase is a continuous phase. Thus, the composition according to the present invention may be in the form of O/W (oil-in-water).

The (a) cationic polymer can be hydrophobized by the (f) fatty acid. The hydrophobized (a) cationic polymer can be present between the fatty phase and the aqueous phase to stabilize the fatty phase. Thus, the fatty phases can be stably dispersed in the aqueous phase.

The composition according to the present invention is stable even when it comprises a relatively large amount of the (e) oil. Thus, the composition according to the present invention can comprise a relatively large amount of the (e) oil.

The composition according to the present invention is stable for a long period of time. In other words, the phase separation of the composition according to the present invention can be prevented for a long period of time.

Accordingly, the composition according to the present invention can be stored for a long period of time.

Hereinafter, the present invention will be explained in a more detailed manner.

### [Composition]

### (Cationic Polymer)

The composition according to the present invention comprises (a) at least one cationic polymer.

There is no limit to the type of the (a) cationic polymer. Two or more different types of cationic polymers may be used in combination. Thus, a single type of cationic polymer or a combination of different types of cationic polymers may be used.

A cationic polymer has a positive charge density. The charge density of the (a) cationic polymer may be from 0.01 meq/g to 20 meq/g, preferably from 0.05 to 15 meq/g, and more preferably from 0.1 to 10 meq/g.

The (a) cationic polymer is selected from chitosans with a molecular weight (Da) of more than 20,000, preferably more than 30,000, more preferably more than 50,000, and even more preferably more than 80,000. In other words, the (a) cationic polymer is a high molecular weight chitosan.

The molecular weight (Da) of the (a) cationic polymer may be less than 2,000,000, preferably less than 1,000,000, more preferably less than 500,000, and even more preferably less than 300,000.

Thus, the molecular weight (Da) of the (a) cationic polymer may be more than 20,000 and less than 2,000,000, preferably more than 30,000 and less than 1,000,000, more preferably more than 50,000 and less than 500,000, and even more preferably more than 80,000 and less than 300,000.

Unless otherwise defined in the descriptions, “molecular weight” means a weight average molecular weight. The molecular weight can be measured or determined by a gel permeation chromatography, for example, in accordance with ASTM D5296-19.

Chitosan is very uncommon in nature. It is only reported in the exoskeletons of certain insects such as termite queens and in the cell walls of a particular class of fungi, zygomycetes.

Chitosan may be obtained by deacetylation of chitin. Chitin is a polysaccharide composed of several N-acetyl-D-glucosamine units linked together by a type β bond (1,4).

The ideal chemical structure of chitosan is a sequence of 3-D-glucosamine monomers connected by a glycosidic bond (1→4).

“Chitosan” according to the present invention means any copolymer formed of constituent units N-acetyl-D-glucosamine and D-glucosamine, whose degree of acetylation is less than 90%, preferably less than 80%, preferably less than 70%, preferably less than 60%, preferably less than 50%. Chitosan consists of glucosamine sugar units (deacetylated units) and N-acetyl-D-glucosamine units (acetylated units) linked together by β type bonds (1,4) and is a polymer of the Poly (N-acetyl-D-glucosamine)-poly (D-glucosamine) type.

More preferably, the degree of acetylation of chitosan is less than or equal to 40%, preferably less than or equal to 35%, preferably less than or equal to 25%, preferably less than or equal to 15%, and preferably less than or equal to 10%.

The degree of acetylation is the percentage of acetylated units relative to the number of total units, it can be determined by Fourier transform infrared spectroscopy (FT-IR) or by titration by a strong base.

The chitosan of the present invention is preferably a polysaccharide prepared from a fungal origin. In particular, it is extracted and purified from safe and abundant food or biotechnological fungal sources such as Agaricus bisporus or Aspergillus niger.

The chitosan of the present invention is preferably derived from the mycelium of a fungus of the Ascomycete type, and in particular Aspergillus niger and/or a Basidiomycete fungus, and in particular Lentinula edodes (shiitake) and/or Agaricus bisporus. Preferably the fungus is Aspergillus niger.

Chitosan may be of genetically modified organisms (GMO) origin, but preferably is of non-GMO origin.

The chitosan according to the present invention is native, that is to say that it is not modified. In particular, it does not contain any chemical modification.

One method of preparing chitosan is that described in WO03/068824.

Preferably, the chitosan used in the present invention is in a powder form. It is marketed by Kitozyme under the name Kiosmetine or Kionutrime, for example, Kiosmetine-CSH and Kiosmetine P.

The amount of the (a) cationic polymer(s) in the composition according to the present invention may be 0.01% by weight or more, preferably 0.05% by weight or more, and more preferably 0.1% by weight or more, relative to the total weight of the composition.

The amount of the (a) cationic polymer(s) in the composition according to the present invention may be 15% by weight or less, preferably 10% by weight or less, and more preferably 5% by weight or less, relative to the total weight of the composition.

The amount of the (a) cationic polymer(s) in the composition according to the present invention may be from 0.01% to 15% by weight, preferably from 0.05% to 10% by weight, and more preferably from 0.1% to 5% by weight, relative to the total weight of the composition.

### (Monovalent Non-Polymeric Acid or Salt Thereof)

The composition according to the present invention comprises (b) at least one monovalent non-polymeric acid or a salt thereof. Two or more monovalent non-polymeric acid or salts thereof may be used in combination. Thus, a single type of monovalent non-polymeric acid or a salt thereof or a combination of different types of monovalent non-polymeric acids or salts thereof may be used.

The term “non-polymeric” here means that the acid is not obtained by polymerizing two or more monomers. Therefore, the non-polymeric acid does not correspond to an acid obtained by polymerizing two or more monomers such as polyacrylic acids.

The term “salt” here means a salt formed by addition of suitable base(s) to the monovalent non-polymeric acid, which may be obtained from a reaction with the monovalent non-polymeric acid with the base(s) according to methods known to those skilled in the art. As the salt, mention may be made of metal salts, for example salts with alkaline metal such as Na and K, and salts with alkaline earth metal such as Mg and Ca, and ammonium salts.

It is preferable that the molecular weight of the (b) monovalent non-polymeric acid or salt thereof be less than 1000, preferably 500 or less, and more preferably 100 or less.

The (b) monovalent non-polymeric acid or a salt thereof can be included in the aqueous phase formed by the (d) water. The (b) monovalent non-polymeric acid or a salt thereof may promote the dissolution of the (a) cationic polymer in the aqueous phase.

The (b) monovalent non-polymeric acid or a salt thereof may be selected from monovalent organic or inorganic acids and salts thereof, preferably monovalent organic acids and salts thereof, and more preferably monovalent carboxylic acids and salts thereof.

The monovalent non-polymeric acid has a single acid group which may be selected from the group consisting of a carboxylic group, a sulfuric group, a sulfonic group, a phosphoric group, a phosphonic group, and a mixture thereof.

The monovalent non-polymeric acid may be selected from hydroxyl acids, and preferably alpha-hydroxy acids. As the alpha-hydroxy acids, mention may be made of, for example, lactic acid and glycolic acid.

The monovalent non-polymeric acid may be a monovalent non-polymeric organic acid, preferably a monovalent carboxylic acid, and more preferably lactic acid.

The amount of the (b) monovalent non-polymeric acid(s) or salt(s) thereof in the composition according to the present invention may be 0.01% by weight or more, preferably 0.05% by weight or more, and more preferably 0.1% by weight or more, relative to the total weight of the composition.

The amount of the (b) monovalent non-polymeric acid(s) or salt(s) thereof in the composition according to the present invention may be 15% by weight or less, preferably 10% by weight or less, and more preferably 5% by weight or less, relative to the total weight of the composition.

The amount of the (b) monovalent non-polymeric acid(s) or salt(s) thereof in the composition according to the present invention may be from 0.01% to 15% by weight, preferably from 0.05% to 10% by weight, and more preferably from 0.1% to 5% by weight, relative to the total weight of the composition.

### (Non-Polymeric Acid Having Two or More Acid Dissociation Constants)

The composition according to the present invention comprises (c) at least one non-polymeric acid having two or more pKa values or a salt thereof, i.e., at least one non-polymeric acid having two or more acid dissociation constants or a salt thereof. The pKa value (acid dissociation constant) is well known to those skilled in the art, and should be determined at a constant temperature such as 25° C.

The (c) non-polymeric acid having two or more pKa values or a salt thereof can be included in the aqueous phase including (d) water. The non-polymeric acid having two or more pKa values can function as a crosslinker for the (a) cationic polymer.

The term “non-polymeric” here means that the acid is not obtained by polymerizing two or more monomers. Therefore, the non-polymeric acid does not correspond to an acid obtained by polymerizing two or more monomers, such as polyacrylic acid.

It is preferable that the molecular weight of the (c) non-polymeric acid having two or more pKa values or a salt thereof is 1000 or less, preferably 800 or less, and more preferably 700 or less.

There is no limit to the type of the (c) non-polymeric acid having two or more pKa values or a salt thereof. Two or more different types of (c) non-polymeric acids having two or more pKa values or salts thereof may be used in combination. Thus, a single type of a (c) non-polymeric acid having two or more pKa values or a salt thereof or a combination of different types of (c) non-polymeric acids having two or more pKa values or salts thereof may be used.

The term “salt” here means a salt formed by addition of suitable base(s) to the non-polymeric acid having two or more pKa values, which may be obtained from a reaction with the non-polymeric acid having two or more pKa values with the base(s) according to methods known to those skilled in the art. As the salt, mention may be made of metal salts, for example salts with alkaline metal such as Na and K, and salts with alkaline earth metal such as Mg and Ca, and ammonium salts.

The (c) non-polymeric acid having two or more pKa values or a salt thereof may be an organic acid, an inorganic acid, or a salt thereof, and preferably a hydrophilic or water-soluble organic acid, an inorganic acid, or a salt thereof.

The non-polymeric acid having two or more pKa values may have at least two monovalent acid groups or at least one polyvalent acid group, which may be selected from the group consisting of a carboxylic group, a sulfuric group, a sulfonic group, a phosphoric group, a phosphonic group, a phenolic hydroxyl group, and a mixture thereof.

It is preferable that non-polymeric acid having two or more pKa values have at least one phosphoric group and/or at least two carboxylic acid groups.

The non-polymeric acid having two or more pKa values may be a non-polymeric polyvalent acid. The non-polymeric polyvalent acid may be selected from the group consisting of dicarboxylic acids, disulfonic acids, phosphoric acid, diphosphoric acids, and a mixture thereof.

The (c) non-polymeric acid having two or more pKa values or a salt thereof may be selected from the group consisting of oxalic acid, malonic acid, succinic acid, glutaric acid, adipic acid, pimelic acid, suberic acid, azelaic acid, sebacic acid, fumaric acid, maleic acid, malic acid, citric acid, aconitic acid, oxaloacetic acid, tartaric acid, and salts thereof, aspartic acid, glutamic acid, and salts thereof; terephthalylidene dicamphor sulfonic acid or salts thereof (Mexoryl SX), Benzophenone-9; phytic acid, and salts thereof, Red 2 (Amaranth), Red 102 (New Coccine), Yellow 5 (Tartrazine), Yellow 6 (Sunset Yellow FCF), Green 3 (Fast Green FCF), Blue 1 (Brilliant Blue FCF), Blue 2 (Indigo Carmine), Red 201 (Lithol Rubine B), Red 202 (Lithol Rubine BCA), Red 204 (Lake Red CBA), Red 206 (Lithol Red CA), Red 207 (Lithol Red BA), Red 208 (Lithol Red SR), Red 219 (Brilliant Lake Red R), Red 220 (Deep Maroon), Red 227 (Fast Acid Magenta), Yellow 203 (Quinoline Yellow WS), Green 201 (Alizanine Cyanine Green F), Green 204 (Pyranine Conc), Green 205 (Light Green SF Yellowish), Blue 203 (Patent Blue CA), Blue 205 (Alfazurine FG), Red 401 (Violamine R), Red 405 (Permanent Re F5R), Red 502 (Ponceau 3R), Red 503 (Ponceau R), Red 504 (Ponceau SX), Green 401 (Naphtol Green B), Green 402 (Guinea Green B), and Black 401 (Naphtol Blue Black); folic acid, ascorbic acid, erythorbic acid, and salts thereof, cystine and salts thereof, EDTA and salts thereof, glycyrrhizin and salts thereof; and a mixture thereof.

It may be preferable that the (c) non-polymeric acid having two or more pKa values or a salt thereof be selected from the group consisting of phosphoric acid, diphosphoric acid, phosphonic acid, phytic acid, citric acid, tartaric acid, and salts thereof, and more preferably phosphoric acid, diphosphoric acid, phytic acid, citric acid, tartaric acid, and salts thereof.

The amount of the (c) non-polymeric acid(s) having two or more pKa values or salt(s) thereof in the composition according to the present invention may be 0.001% by weight or more, preferably 0.003% by weight or more, and more preferably 0.005% by weight or more, relative to the total weight of the composition.

The amount of the (c) non-polymeric acid(s) having two or more pKa values or salt(s) thereof in the composition according to the present invention may be 10% by weight or less, preferably 5% by weight or less, and more preferably 1% by weight or less, relative to the total weight of the composition.

The amount of the (c) non-polymeric acid(s) having two or more pKa values or salt(s) thereof in the composition according to the present invention may be from 0.001% to 10% by weight, preferably from 0.003% to 5% by weight, and more preferably from 0.005% to 1% by weight, relative to the total weight of the composition.

### (Water)

Preferably, the composition according to the present invention comprises (d) water.

The (d) water can constitute an aqueous phase which can be, for example, a continuous phase in the composition according to the present invention.

The amount of the (d) water may be 50% by weight or more, preferably 60% by weight or more, and more preferably 70% by weight or more, relative to the total weight of the composition.

The amount of the (d) water may be 99% by weight or less, preferably 97% by weight or less, and more preferably 95% by weight or less, relative to the total weight of the composition.

The amount of the (d) water may be from 50% to 99% by weight, preferably from 60% to 97% by weight, and more preferably from 70% to 95% by weight, relative to the total weight of the composition.

### (Oil)

The composition according to the present invention may comprise (e) at least one oil. If two or more oils are used, they may be the same or different.

The (e) oil can constitute fatty phases which may be, for example, dispersed or discontinuous phases in the composition according to the present invention.

Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

The (e) oil may be a non-polar oil such as a hydrocarbon oil, a silicone oil, or the like; a polar oil such as a plant or animal oil and an ester oil or an ether oil; or a mixture thereof.

The (e) oil may be selected from the group consisting of oils derived from biological resources such as oils of plant or animal origin; oils derived from non-biological resources such as synthetic oils, silicone oils, hydrocarbon oils and fatty alcohols, derived from chemicals; and mixtures thereof.

As examples of plant oils, mention may be made of, for example, apricot oil, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

As examples of animal oils, mention may be made of, for example, squalene and squalane.

As examples of synthetic oils, mention may be made of alkane oils such as isododecane and isohexadecane, ester oils, ether oils, and artificial triglycerides.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the present invention are derived is branched.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate and isostearyl neopentanoate.

Esters of C₄-C₂₂ dicarboxylic or tricarboxylic acids and of C₁-C₂₂ alcohols, and esters of monocarboxylic, dicarboxylic or tricarboxylic acids and of non-sugar C₄-C₂₆ dihydroxy, trihydroxy, tetrahydroxy or pentahydroxy alcohols may also be used.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; and diethylene glycol diisononanoate.

As ester oils, one can use sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from monoesters, diesters, triesters, tetraesters and polyesters, and mixtures thereof.

These esters may be, for example, oleates, laurates, palmitates, myristates, behenates, cocoates, stearates, linoleates, linolenates, caprates and arachidonates, or mixtures thereof such as, especially, oleopalmitate, oleostearate and palmitostearate mixed esters, as well as pentaerythrityl tetraethyl hexanoate.

More particularly, use is made of monoesters and diesters and especially sucrose, glucose or methylglucose monooleates or dioleates, stearates, behenates, oleopalmitates, linoleates, linolenates and oleostearates.

An example that may be mentioned is the product sold under the name Glucate® DO by the company Amerchol, which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrityl tetra(2-ethylhexanoate), 2-ethylhexyl succinate, diethyl sebacate, and mixtures thereof.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate) and glyceryl tri(caprate/caprylate/linolenate).

Hydrocarbon Oils May be Chosen from:
linear or branched, optionally cyclic, C₆-C₁₆ lower alkanes. Examples that may be mentioned include hexane, undecane, dodecane, tridecane, and isoparaffins, for instance isohexadecane, isododecane and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosan, and decene/butene copolymer; and mixtures thereof.

The term “fatty” in the fatty alcohol means the inclusion of a relatively large number of carbon atoms. Thus, alcohols which have 4 or more, preferably 6 or more, and more preferably 12 or more carbon atoms are encompassed within the scope of fatty alcohols. The fatty alcohol may be saturated or unsaturated. The fatty alcohol may be linear or branched.

The fatty alcohol may have the structure R—OH wherein R is chosen from saturated and unsaturated, linear and branched radicals containing from 4 to 40 carbon atoms, preferably from 6 to 30 carbon atoms, and more preferably from 12 to 20 carbon atoms. In at least one embodiment, R may be chosen from C₁₂-C₂₀ alkyl and C₁₂-C₂₀ alkenyl groups. R may or may not be substituted with at least one hydroxyl group.

As examples of the fatty alcohol, mention may be made of lauryl alcohol, cetyl alcohol, stearyl alcohol, isostearyl alcohol, behenyl alcohol, undecylenyl alcohol, myristyl alcohol, octyldodecanol, hexyldecanol, oleyl alcohol, linoleyl alcohol, palmitoleyl alcohol, arachidonyl alcohol, erucyl alcohol, and mixtures thereof.

It is preferable that the fatty alcohol be a saturated fatty alcohol.

Thus, the fatty alcohol may be selected from straight or branched, saturated or unsaturated C₆-C₃₀ alcohols, preferably straight or branched, saturated C₆-C₃₀ alcohols, and more preferably straight or branched, saturated C₁₂-C₂₀ alcohols.

The term “saturated fatty alcohol” here means an alcohol having a long aliphatic saturated carbon chain. It is preferable that the saturated fatty alcohol be selected from any linear or branched, saturated C₆-C₃₀ fatty alcohols. Among the linear or branched, saturated C₆-C₃₀ fatty alcohols, linear or branched, saturated C₁₂-C₂₀ fatty alcohols may preferably be used. Any linear or branched, saturated C₁₆-C₂₀ fatty alcohols may be more preferably used. Branched C₁₆-C₂₀ fatty alcohols may be even more preferably used.

As examples of saturated fatty alcohols, mention may be made of lauryl alcohol, cetyl alcohol, stearyl alcohol, isostearyl alcohol, behenyl alcohol, undecylenyl alcohol, myristyl alcohol, octyldodecanol, hexyldecanol, and mixtures thereof. In one embodiment, cetyl alcohol, stearyl alcohol, octyldodecanol, hexyldecanol, or a mixture thereof (e.g., cetearyl alcohol) as well as behenyl alcohol, can be used as a saturated fatty alcohol.

According to at least one embodiment, the fatty alcohol used in the composition according to the present invention is preferably chosen from octyldodecanol, hexyldecanol and mixtures thereof.

It is preferable to use oils from derived from biological resources such as oils of plant or animal origin, and more preferably plant oils.

The oils of plant or animal origin may include hydrocarbon oils.

As preferable examples of hydrocarbon oils from plants, mention may be made of, for example, linear or branched hydrocarbons such as dodecane, tetradecane, hexadecane, octadecane, squalane, hemisqualane, and the like.

It is preferable that the (e) oil be selected from plant oils, synthetic ester oils, and mixtures thereof, and preferably from plant oils.

If present, the (e) oil may be surrounded by a plurality of the (a) cationic polymers or a complex of the (a) cationic polymers and the (c) non-polymeric acid having two or more pKa values or a salt thereof, or the (e) oil may be present in the hollow of a capsule formed by the (a) cationic polymers or the above complex. In other words, the (e) oil may be covered by the (a) cationic polymers or the above complex, or a capsule formed by the (a) cationic polymers and the above complex includes the (e) oil in the hollow of the capsule.

The (e) oil which is surrounded by the (a) cationic polymers or the above complex, or present in the hollow of the capsule formed by the (a) cationic polymers or the above complex cannot directly make contact with keratin fibers such as hair. Thus, even if the (e) oil has a sticky or greasy feeling of use, the composition according to the present invention would not provide a sticky or greasy feeling of use.

The amount of the (e) oil(s) in the composition according to the present invention may be 1% by weight or more, preferably 3% by weight or more, and more preferably 5% by weight or more, relative to the total weight of the composition.

The amount of the (e) oil(s) in the composition according to the present invention may be 50% by weight or less, preferably 45% by weight or less, and more preferably 40% by weight or less, relative to the total weight of the composition.

The amount of the (e) oil(s) in the composition according to the present invention may be from 1% to 50% by weight, preferably from 3% to 45% by weight, and more preferably from 5% to 40% by weight, relative to the total weight of the composition.

### (Fatty Acid)

The composition according to the present invention may comprise (f) at least one fatty acid. If two or more fatty acids are used, they may be the same or different.

The term “fatty acid” here means a carboxylic acid with a long aliphatic carbon chain.

The (f) fatty acid has at least 4 carbon atoms, preferably at least 6 carbon atoms, and more preferably at least 8 carbon atoms. The (f) fatty acid may comprise up to 26 carbon atoms, preferably up to 24 carbon atoms, and more preferably up to 22 carbon atoms. It is preferable that the (f) fatty acid be selected from C₄-C₂₆ fatty acid, more preferably C₆-C₂₄ fatty acid, and even more preferably C₈-C₂₂ fatty acid.

The (f) fatty acid may be selected from saturated or unsaturated, linear or branched fatty acids. Thus, the (f) fatty acid may be selected from C₄-C₂₆, preferably C₆-C₂₄, more preferably C₈-C₂₂ saturated and unsaturated, linear or branched fatty acids.

As the unsaturated, linear or branched fatty acids, mono-unsaturated, linear or branched fatty acids or polyunsaturated, linear or branched fatty acids may be used. As the unsaturated moiety of the unsaturated, linear or branched fatty acids, a carbon-carbon double bond or a carbon-carbon triple bond may be mentioned.

As the saturated fatty acid, mention may be made of, for example, caprylic acid (C₈), pelargonic acid (C₉), capric acid (C₁₀), lauric acid (C₁₂), myristic acid (C₁₄), pentadecanoic acid (C₁₅), palmitic acid (C₁₆), heptadecanoic acid (C₁₇), stearic acid (C₁₈), isostearic acid (C₁₈), nonadecanoic acid (C₁₉), arachidic acid (C₂₀), behenic acid (C₂₂), and lignoceric acid (C₂₄).

As the unsaturated fatty acid, mention may be made of, for example, myristoleic acid (C₁₄), palmitoleic acid (C₁₆), oleic acid (C₁₈), linoleic acid (C₁₈), linolenic acid (C₁₈), elaidic acid (C₁₈), arachidonic acid (C₂₀), eicosenoic acid (C₂₀), erucic acid (C₂₂), and nervonic acid (C₂₄).

It is preferable that the (f) fatty acid be selected from C₈-C₁₈ saturated or unsaturated, linear or branched fatty acids, and more preferably from the group consisting of caprylic acid, capric acid, oleic acid, linoleic acid, stearic acid, isostearic acid and mixtures thereof.

The (f) fatty acid may be in the form of a free acid or in the form of a salt thereof. As a salt of the fatty acid, mention may be made of an inorganic salt such as an alkali metal salt (a sodium salt, a potassium salt, or the like) and an alkaline earth metal salt (a magnesium salt, a calcium salt, or the like); and an organic salt such as an ammonium salt (a quaternary ammonium salt or the like) and an amine salt (a triethanolamine salt, a triethylamine salt, or the like). A single type of fatty acid salt or a combination of different type of fatty acid salts may be used. Further, a combination of one or more fatty acid in the form of a free acid and one or more fatty acid in the form of a salt may be used, in which one or more type of salts may also be used.

The amount of the (f) fatty acid(s) in the composition according to the present invention may be 0.01% by weight or more, preferably 0.05% by weight or more, and more preferably 0.1% by weight or more, relative to the total weight of the composition. It may be even more preferable that the amount of the (f) fatty acid(s) in the composition according to the present invention be 1% by weight or more, relative to the total weight of the composition.

On the other hand, the amount of the (f) fatty acid(s) in the composition according to the present invention may be 15% by weight or less, preferably 10% by weight or less, and more preferably 5% by weight or less, relative to the total weight of the composition. It may be even more preferable that the amount of the (f) fatty acid(s) in the composition according to the present invention be 4% by weight or less, relative to the total weight of the composition.

Accordingly, the amount of the (f) fatty acid(s) in the composition according to the present invention may range from 0.01% to 15% by weight, preferably from 0.05% to 10% by weight, and more preferably from 0.1% to 5% by weight, relative to the total weight of the composition. It may be even more preferable that the amount of the (f) fatty acid(s) in the composition according to the present invention be from 1% to 4% by weight, relative to the total weight of the composition.

(pH)

The pH of the composition according to the present invention may be from 3 to 9, preferably from 3.3 to 8.5, and more preferably from 3.5 to 8.

At a pH of from 3 to 9, the (a) cationic polymer or a complex of the (a) cationic polymer and the (c) non-polymeric acid having two or more pKa values or a salt thereof can be very stable.

Thus, the composition according to the present invention can be very stable under the pH of from 3 to 9.

The pH of the composition according to the present invention may be adjusted by adding at least one alkaline agent and/or at least one acid, other than the (b) monovalent non-polymeric acid or a salt thereof or the (c) non-polymeric acid having two or more pKa values or a salt thereof. The pH of the composition according to the present invention may also be adjusted by adding at least one buffering agent.

### (Alkaline Agent)

The composition according to the present invention may comprise at least one alkaline agent.

Two or more alkaline agents may be used in combination. Thus, a single type of alkaline agent or a combination of different types of alkaline agents may be used.

The alkaline agent may be an inorganic alkaline agent. It is preferable that the inorganic alkaline agent be selected from the group consisting of ammonia; alkaline metal hydroxides; alkaline earth metal hydroxides; alkaline metal phosphates and monohydrogenophosphates such as sodium phosphate or sodium monohydrogen phosphate.

As examples of the inorganic alkaline metal hydroxides, mention may be made of sodium hydroxide and potassium hydroxide. As examples of the alkaline earth metal hydroxides, mention may be made of calcium hydroxide and magnesium hydroxide. As an inorganic alkaline agent, sodium hydroxide is preferable.

The alkaline agent may be an organic alkaline agent. It is preferable that the organic alkaline agent be selected from the group consisting of monoamines and derivatives thereof, diamines and derivatives thereof, polyamines and derivatives thereof; basic amino acids and derivatives thereof, oligomers of basic amino acids and derivatives thereof; polymers of basic amino acids and derivatives thereof, urea and derivatives thereof, and guanidine and derivatives thereof.

As examples of the organic alkaline agents, mention may be made of alkanolamines such as mono-, di- and tri-ethanolamine, and isopropanolamine; urea, guanidine and their derivatives; basic amino acids such as ornithine; and diamines such as those described in the structure below:

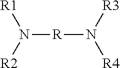

wherein R denotes an alkylene such as propylene optionally substituted by a hydroxyl or a C₁-C₄ alkyl radical, and R₁, R₂, R₃ and R₄ independently denote a hydrogen atom, an alkyl radical or a C₁-C₄ hydroxyalkyl radical, which may be exemplified by 1,3-propanediamine and derivatives thereof.

The alkaline agent(s) may be used in a total amount of from 0.01% to 15% by weight, preferably from 0.02% to 10% by weight, more preferably from 0.03% to 5% by weight, relative to the total weight of the composition, depending on their solubility.

### (Acid)

The composition according to the present invention may comprise at least one acid. Two or more acids may be used in combination. Thus, a single type of acid or a combination of different types of acids may be used.

As the acid, mention may be made of any inorganic or organic acids, preferably inorganic acids, which are commonly used in cosmetic products. A monovalent acid and/or a polyvalent acid may be used. A monovalent acid such as hydrochloric acid (HCl) may be used.

The acid(s) may be used in a total amount of from 0.01% to 15% by weight, preferably from 0.02% to 10% by weight, more preferably from 0.03% to 5% by weight, relative to the total weight of the composition, depending on their solubility.

### (Optional Ingredient)

The composition according to the present invention may comprise, in addition to the aforementioned ingredients, optional ingredient(s) typically employed in cosmetics, specifically, surfactants/emulsifiers, hydrophilic or lipophilic thickeners, derived from, for example, synthetic polymers other than the (a) cationic polymer; volatile or non-volatile organic solvents; anionic polymers; amphoteric polymers; nonionic polymers such as beta-glucan; silicones and silicone derivatives other than the (e) oil; natural extracts derived from animals or vegetables other than the (a) cationic polymer or the (e) oil; waxes; and the like, within a range which does not impair the effects of the present invention.

The composition according to the present invention may comprise the above optional ingredient(s) in an amount of from 0.01% to 30% by weight, preferably from 0.05% to 20% by weight, and more preferably from 0.1% to 10% by weight, relative to the total weight of the composition.

The amount of the surfactant(s)/emulsifier(s) and/or synthetic thickener(s) and/or organic solvent(s) in the composition according to the present invention may be 1% by weight or less, preferably 0.1% by weight or less, and more preferably 0.01% by weight or less, relative to the total weight of the composition. It is particularly preferable that the composition according to the present invention includes no surfactant/emulsifier or synthetic thickener or organic solvent.

### [Preparation]

The composition according to the present invention can be prepared by mixing the essential ingredient(s) as explained above, and optional ingredient(s), if necessary, as explained above.

The method and means to mix the above essential and optional ingredients are not limited. Any conventional method and means can be used to mix the above essential and optional ingredients to prepare the composition according to the present invention.

The composition according to the present invention can be prepared by simple or easy mixing with a conventional mixing means such as a stirrer and a homogenizer. Also, heating may not be necessary. Therefore, the process for preparing the composition according to the present invention may be environmentally friendly.

### [Cosmetic Application]

The composition according to the present invention may be intended to be used as a cosmetic composition.

The cosmetic composition according to the present invention may be intended for application onto keratin fibers. Keratin fibers here means fibers containing keratin as a main constituent element, and examples thereof include hair and the like. It is preferable that the cosmetic composition according to the present invention be used for a cosmetic process for keratin fibers, in particular hair.

It is preferable that the cosmetic composition according to the present invention be a cosmetic composition for keratin fibers such as hair, and more preferably a hair care cosmetic composition.

### [Form]

The composition according to the present invention may be present in any form.

If the composition according to the present invention includes (e) oil, the (e) oil can form fatty phases, the (d) water can form an aqueous phase, and the fatty phases can be dispersed in the aqueous phase. Thus, the aqueous phase can function as a continuous phase, and the fatty phase can function as a dispersed phase. In other words, the composition according to the present invention may be in the form of an O/W dispersion such as an O/W emulsion. If the composition according to the present invention is of the O/W type, it can provide a fresh sensation due to the (d) water which forms the outer phase thereof.

### [Mechanism]

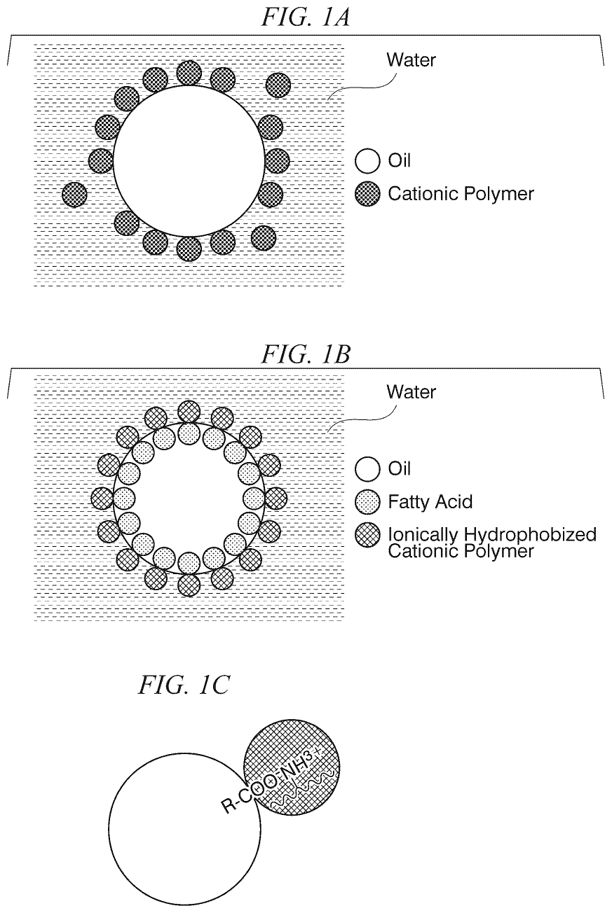

FIG. 1 shows schematic drawings showing the behaviors of the (a) cationic polymers around a fatty phase, if present, such as an oil droplet, dispersed in an aqueous phase, and the examples of hydrophobicization of the (a) cationic polymer, in one embodiment of the present invention.

A plurality of the (a) cationic polymers or a complex of the (a) cationic polymers and the (c) non-polymeric acid having two or more pKa values or a salt thereof can be present at the interface between the fatty phase and the aqueous phase. Thus, the (a) cationic polymers or the above complex can form an emulsion without the aid by any conventional surfactant or emulsifier. The emulsion formed by the (a) cationic polymer or the above complex may be similar to a so-called Pickering emulsion.

Alternatively, a plurality of the (a) cationic polymers or the above complex can form a capsule having a hollow. The (e) oil can be present in the hollow. In other words, the (e) oil can be incorporated into the capsule. The wall of the capsule may be composed of a continuous layer or film formed from the (a) cationic polymers or the above complex. While not wishing to be bound by theory, it is believed that the (a) cationic polymers or the above complex can re-organize at the interface of the (e) oil and the (d) water to spontaneously form a capsule having a hollow to include the (e) oil. For example, a continuous aqueous phase comprising the (d) water and dispersed phases comprising the (e) oil in the capsule can form an O/W emulsion which may also be similar to a so-called Pickering emulsion.

The fatty phase can comprise the (f) fatty acid, if present. The (f) fatty acid in the fatty phase can hydrophobize the (a) cationic polymer in-situ. FIG. 1 shows a scheme of hydrophobization of the (a) cationic polymer by the (f) fatty acid

FIG. 1A shows a schematic drawing showing the behaviour of the (a) cationic polymers around a fatty phase, if present, such as an oil droplet, dispersed in an aqueous phase, in case of the fatty phase including no (f) fatty acid.

In FIG. 1A, the (a) cationic polymer may have insufficient hydrophobicity. Therefore, the affinity between the (a) cationic polymer and the fatty phase including the (e) oil may be limited. Thus, the emulsification of the fatty phase in the aqueous phase may be less stable.

On the other hand, FIG. 1B shows a schematic drawing showing the behaviour of the (a) cationic polymers around a fatty phase, such as an oil droplet, dispersed in an aqueous phase, in case of the fatty phase including (f) fatty acid.

In FIG. 1B, the carboxylic group in the (f) fatty acid in the fatty phase can ionically interact with the cationic or cationizable group, such as an ammonium group or amino group, in the (a) cationic polymer, as shown in FIG. 1C. Accordingly, the (a) cationic polymer is ionically hydrophobized and can have sufficient hydrophobicity. Therefore, the affinity between the (a) cationic polymer and the fatty phase including the (e) oil is enhanced. Thus, the emulsification of the fatty phase in the aqueous phase is stable.

### [Film]

The composition according to the present invention can be used for easily preparing a film.

Thus, the present invention may also relate to a process for preparing a film, preferably a cosmetic film, optionally with a thickness of preferably more than 0.5 μm, more preferably 1.0 μm or more, and even more preferably 1.5 μm or more, comprising:
applying onto keratin fibers such as hair the composition according to the present invention; and
drying the composition.

The upper limit of the thickness of the film according to the present invention is not limited. Thus, for example, the thickness of the film according to the present invention may be 1 mm or less, preferably 500 μm or less, more preferably 300 μm or less, and even more preferably 100 μm or less.

Since the process for preparing a film according to the present invention includes the steps of applying the composition according to the present invention onto keratin fibers such as hair, and of drying the composition, the process according to the present invention does not require any spin coating or spraying, and therefore, it is possible to easily prepare even a relatively thick film. Thus, the process for preparing a film according to present invention can prepare a relatively thick film without any special equipment such as spin coaters and spraying machines.

Even if the film according to the present invention is relatively thick, it is still thin and may be transparent, and therefore, may not be easy to perceive. Thus, the film according to the present invention can be used preferably as a cosmetic film.

The present invention may also relate to:

(1) A film, preferably a cosmetic film, optionally with a thickness of preferably more than 0.5 μm, more preferably 1.0 μm or more, and even more preferably 1.5 μm or more, prepared by a process comprising:
applying onto keratin fibers such as hair the composition according to the present invention; and
drying the composition,
   and
   (2) A film, preferably a cosmetic film, optionally with a thickness of preferably more than 0.5 μm, more preferably 1.0 μm or more, and even more preferably 1.5 μm or more, comprising:
(a) at least one cationic polymer selected from chitosans with a molecular weight of more than 20,000;
(b) at least one monovalent non-polymeric acid or a salt thereof; and
(c) at least one non-polymeric acid having two or more pKa values or a salt thereof.

The film may also include (e) at least one oil and/or (f) at least one fatty acid.

The above explanations regarding the (a) cationic polymer, the (b) monovalent non-polymeric acid or a salt thereof, and the (c) non-polymeric acid having two or more pKa values or a salt thereof, as well as optional ingredients such as the (e) oil and the (f) the fatty acid, can apply to those in the above films (1) and (2).

It is preferable that the film according to the present invention be hydrophobic.

The term “hydrophobic” in the present specification means that the solubility of the film in water (preferably with a volume of 1 liter) at from 20 to 40° C., preferably from 25 to 40° C., and more preferably from 30 to 40° C. is less than 10% by weight, preferably less than 5% by weight, more preferably less than 1% by weight, and even more preferably less than 0.1% by weight, relative to the total weight of the film. It is most preferable that the film is not soluble in water.

If the film according to the present invention is hydrophobic, the film can have water-resistant properties, and therefore, it can remain on keratin fibers such as hair even if the surface of the keratin fibers is wet due to, for example, sweat and rain. Thus, when the film according to the present invention provides any cosmetic effect, the cosmetic effect can last a long time.

The film according to the present invention may comprise at least one biocompatible and/or biodegradable polymer layer. Two or more biocompatible and/or biodegradable polymers may be used in combination. Thus, a single type of biocompatible and/or biodegradable polymer or a combination of different types of biocompatible and/or biodegradable polymers may be used.

The term “biocompatible” polymer in the present specification means that the polymer does not have excess interaction between the polymer and cells in the living body including the skin, and the polymer is not recognized by the living body as a foreign material.

The term “biodegradable” polymer in the present specification means that the polymer can be degraded or decomposed in a living body due to, for example, the metabolism of the living body itself or the metabolism of the microorganisms which may be present in the living body. Also, the biodegradable polymer can be degraded by hydrolysis.

If the film according to the present invention includes a biocompatible and/or biodegradable polymer, for example, it may be less irritable or not irritable to the scalp and/or it may not contaminate environments.

In fact, the film according to the present invention can include (a) at least one cationic polymer selected from chitosans which are biodegradable polymers.

The film according to the present invention can be used for cosmetic treatments of keratin fibers such as hair.

### [Cosmetic Process and Use]

The present invention also relates to:
a cosmetic process for keratin fibers such as hair, comprising: applying to the keratin fibers the composition according to the present invention; and drying the composition to form a cosmetic film on the keratin fibers; or
a use of the composition according to the present invention for the preparation of a cosmetic film on keratin fibers such as hair.

The cosmetic process here means a non-therapeutic cosmetic method for caring for and/or coating the surface of keratin fibers such as hair.

The film of the present invention can provide keratin fibers such as hair with good alignment effect and good shape recovery effect, as explained above.

In addition, the above cosmetic film may immediately protect keratin fibers such as hair by covering the surface of the keratin fibers and shielding them, as a barrier, from environmental stresses such as pollutants, contaminants and the like.

The above cosmetic effects can be adjusted or controlled by changing the chemical composition, the thickness and/or the surface roughness of the above cosmetic film.

If the above cosmetic film includes at least one additional cosmetic active ingredient other than the (e) oil, the cosmetic film can have cosmetic effects provided by the additional cosmetic active ingredient(s). For example, if the cosmetic film includes at least one cosmetic active ingredient such as a deodorant agent, the cosmetic film can control odors on keratin fibers such as hair.

The present invention may also relate to a use of (a) at least one cationic polymer selected from chitosans with a molecular weight of more than 20,000, in a composition for treating keratin fibers such as hair, comprising:
(b) at least one monovalent non-polymeric acid or a salt thereof, and
(c) at least one non-polymeric acid having two or more pKa values or a salt thereof, in order to improve alignment and shape recovery of the keratin fibers.

The above composition may also include (e) at least one oil and/or (f) at least one fatty acid.

The above explanations regarding the (a) cationic polymer, the (b) monovalent non-polymeric acid or a salt thereof, and the (c) non-polymeric acid having two or more pKa values or a salt thereof, as well as optional ingredients such as the (d) water, the (e) oil and the (f) fatty acid, can apply to those in the above use.

### EXAMPLES

The present invention will be described in a more detailed manner by way of examples. However, they should not be construed as limiting the scope of the present invention.

### Examples 1-31 and Comparative Examples 1-10

### [Preparations]

Each of the compositions according to Examples 1-31 and Comparative Examples 1-10 was prepared by mixing the ingredients shown in Tables 1-8. The numerical values for the amounts of the ingredients in Tables 1-8 are all based on “% by weight” as raw materials.

The details of the preparation are as follows.

First, chitosan was added to water. Next, lactic acid was added to solubilize chitosan (provided that for Comparative Examples 6-10, lactic acid was not used because the low molecular weight chitosan used therein was water-soluble without the aid of the lactic acid). Then, a crosslinker (phosphoric acid, diphosphoric acid, citric acid, or tartaric acid) was added slowly. Then, fatty acid (oleic acid, isostearic acid, linoleic acid, or erucic acid) was added with an oil (corn oil) to be emulsified and mixed at room temperature.

TABLE 1

| | | | | | | | | Comp. | Comp. |
|---|---|---|---|---|---|---|---|---|---|
| | Ex. 1 | Ex. 2 | Ex. 3 | Ex. 4 | Ex. 5 | Ex. 6 | Ex. 7 | Ex. 1 | Ex. 2 |
| Water | qsp 100 | qsp 100 | qsp 100 | qsp 100 | qsp 100 | qsp 100 | qsp 100 | qsp 100 | qsp 100 |
| Chitosan | 2.0 | 2.0 | 2.0 | 1.0 | 1.0 | 1.0 | 1.0 | 2.0 | 1.0 |
| (M.W. > 20,000) | | | | | | | | | |
| Lactic Acid | 1.0 | 1.0 | 1.0 | 1.0 | 1.0 | 1.0 | 1.0 | 1.0 | 1.0 |
| Phosphoric Acid | 0.2 | 0.4 | 0.6 | 0.1 | 0.2 | 0.3 | 0.4 | — | — |
| Oleic Acid | 1.7 | 1.7 | 1.7 | 1.7 | 1.7 | 1.7 | 1.7 | 1.7 | 1.7 |
| Corn Oil | 5 | 5 | 5 | 5 | 5 | 5 | 5 | 5 | 5 |

Crosslinker: Phosphoric Acid
Fatty Acid: Oleic Acid

TABLE 2

| | Ex. 8 | Ex. 9 | Ex. 10 | Ex. 11 | Ex. 12 | Ex. 13 |
|---|---|---|---|---|---|---|
| Water | qsp 100 | qsp 100 | qsp 100 | qsp 100 | qsp 100 | qsp 100 |
| Chitosan | 2.0 | 2.0 | 2.0 | 1.0 | 1.0 | 1.0 |
| (M.W. > 20,000) | | | | | | |
| Lactic Acid | 1.0 | 1.0 | 1.0 | 1.0 | 1.0 | 1.0 |
| Diphosphoric Acid | 0.1 | 0.2 | 0.3 | 0.1 | 0.2 | 0.3 |
| Oleic Acid | 1.7 | 1.7 | 1.7 | 1.7 | 1.7 | 1.7 |
| Corn Oil | 5 | 5 | 5 | 5 | 5 | 5 |

Crosslinker: Diphosphoric Acid
Fatty Acid: Oleic Acid

TABLE 3

| | Ex. 14 | Ex. 15 | Ex. 16 |
|---|---|---|---|
| Water | qsp 100 | qsp 100 | qsp 100 |
| Chitosan (M.W. >20,000) | 2.0 | 2.0 | 2.0 |
| Lactic Acid | 1.0 | 1.0 | 1.0 |
| Citric Acid | 0.1 | 0.2 | 0.3 |
| Oleic Acid | 1.7 | 1.7 | 1.7 |
| Corn Oil | 5 | 5 | 5 |

Crosslinker: Citric Acid
Fatty Acid: Oleic Acid

TABLE 4

| | Ex. 17 | Ex. 18 | Ex. 19 |
|---|---|---|---|
| Water | qsp 100 | qsp 100 | qsp 100 |
| Chitosan (M.W. >20,000) | 2.0 | 2.0 | 2.0 |
| Lactic Acid | 1.0 | 1.0 | 1.0 |
| Tartaric Acid | 0.1 | 0.2 | 0.3 |
| Oleic Acid | 1.7 | 1.7 | 1.7 |
| Corn Oil | 5 | 5 | 5 |

Crosslinker: Tartaric Acid
Fatty Acid: Oleic Acid

TABLE 5

| | | | | | Comp. |
|---|---|---|---|---|---|
| | Ex. 20 | Ex. 21 | Ex. 22 | Ex. 23 | Ex. 3 |
| Water | qsp 100 | qsp 100 | qsp 100 | qsp 100 | qsp 100 |
| Chitosan | 2.0 | 2.0 | 2.0 | 2.0 | 2.0 |
| (M.W. >20,000) | | | | | |
| Lactic Acid | 1.0 | 1.0 | 1.0 | 1.0 | 1.0 |
| Phosphoric Acid | 0.1 | 0.2 | 0.4 | 0.6 | — |
| Isostearic Acid | 1.7 | 1.7 | 1.7 | 1.7 | 1.7 |
| Corn Oil | 5 | 5 | 5 | 5 | 5 |

Crosslinker: Phosphoric Acid
Fatty Acid: Isostearic Acid

TABLE 6

| | | | | | Comp. |
|---|---|---|---|---|---|
| | Ex. 24 | Ex. 25 | Ex. 26 | Ex. 27 | Ex. 4 |
| Water | qsp 100 | qsp 100 | qsp 100 | qsp 100 | qsp 100 |
| Chitosan | 2.0 | 2.0 | 2.0 | 2.0 | 2.0 |
| (M.W. >20,000) | | | | | |
| Lactic Acid | 1.0 | 1.0 | 1.0 | 1.0 | 1.0 |
| Phosphoric Acid | 0.1 | 0.2 | 0.4 | 0.6 | — |
| Linoleic Acid | 1.7 | 1.7 | 1.7 | 1.7 | 1.7 |
| Corn Oil | 5 | 5 | 5 | 5 | 5 |

Crosslinker: Phosphoric Acid
Fatty Acid: Linoleic Acid

TABLE 7

| | | | | | Comp. |
|---|---|---|---|---|---|
| | Ex. 28 | Ex. 29 | Ex. 30 | Ex. 31 | Ex. 5 |
| Water | qsp 100 | qsp 100 | qsp 100 | qsp 100 | qsp 100 |
| Chitosan | 2.0 | 2.0 | 2.0 | 2.0 | 2.0 |
| (M.W. >20,000) | | | | | |
| Lactic Acid | 1.0 | 1.0 | 1.0 | 1.0 | 1.0 |
| Phosphoric Acid | 0.1 | 0.2 | 0.4 | 0.6 | — |
| Erucic Acid | 1.7 | 1.7 | 1.7 | 1.7 | 1.7 |
| Corn Oil | 5 | 5 | 5 | 5 | 5 |

Crosslinker: Phosphoric Acid
Fatty Acid: Erucic Acid

TABLE 8

| | | Comp. | Comp. | Comp. | Comp. | Comp. |
|---|---|---|---|---|---|---|
| | Ex. 1 | Ex. 6 | Ex. 7 | Ex. 8 | Ex. 9 | Ex. 10 |
| Water | qsp 100 | qsp 100 | qsp 100 | qsp 100 | qsp 100 | qsp 100 |
| Chitosan | 2.0 | — | — | — | — | — |
| (M.W. > 20,000) | | | | | | |
| Chitosan | — | 2.0 | 2.0 | 2.0 | 2.0 | 2.0 |
| (M.W. < 5,000) | | | | | | |
| Lactic Acid | 1.0 | — | — | — | — | — |
| Phosphoric Acid | 0.2 | 0.1 | 0.2 | 0.4 | 0.6 | — |
| Oleic Acid | 1.7 | 1.7 | 1.7 | 1.7 | 1.7 | 1.7 |
| Corn Oil | 5 | 5 | 5 | 5 | 5 | 5 |

Crosslinker: Phosphoric Acid
Fatty Acid: Oleic Acid

### [Evaluations]

### (Hair Alignment)

0.15 g of each of the compositions according to Examples 1-31 and Comparative Examples 1-10 was applied onto 2.7 g of hair, and dried with a hair drier. The hair alignment of the dried hair was visually observed and evaluated in accordance with the following criteria.
Very Good: Much better than the control
Good: Better than the control
Poor: Same as the control

The results are shown in the following Tables 9-14.

### (Shape Recovery)

0.15 g of each of the compositions according to Examples 1-31 and Comparative Examples 1-10 was applied onto 2.7 g of hair, and dried with a hair drier. The dried hair was spread by combing and shape recovery, i.e., how much the hair was aligned back by hand touching, was visually observed and evaluated in accordance with the following criteria.
Very Good: Much better than the control
Good: Better than the control
Poor: Same as the control

The results are shown in the following Tables 9-14.

TABLE 9

| | | | | | | | | | | | | | Comp. |
|---|---|---|---|---|---|---|---|---|---|---|---|---|---|
| | Ex. 1 | Ex. 2 | Ex. 3 | Ex. 8 | Ex. 9 | Ex. 10 | Ex. 14 | Ex. 15 | Ex. 16 | Ex. 17 | Ex. 18 | Ex. 19 | Ex. 1 |
| Hair | Very | Good | Good | Good | Good | Good | Good | Good | Good | Good | Good | Good | Control |
| Alignment | Good | | | | | | | | | | | | |
| Shape | Good | Good | Good | Good | Good | Good | Good | Good | Very | Good | Good | Good | Control |
| Recovery | | | | | | | | | Good | | | | |

High Molecular Weight Chitosan: 2% by weight
Fatty Acid: Oleic Acid

TABLE 10

| | | | | | | | | Comp. |
|---|---|---|---|---|---|---|---|---|
| | Ex. 4 | Ex. 5 | Ex. 6 | Ex. 7 | Ex. 11 | Ex. 12 | Ex. 13 | Ex. 2 |
| Hair | Good | Good | Good | Good | Good | Good | Good | Control |
| Alignment | | | | | | | | |
| Shape | Good | Good | Good | Good | Good | Good | Good | Control |
| Recovery | | | | | | | | |

High Molecular Weight Chitosan: 1% by weight
Fatty Acid: Oleic Acid

TABLE 11

| | | | | | Comp. |
|---|---|---|---|---|---|
| | Ex. 20 | Ex. 21 | Ex. 22 | Ex. 23 | Ex. 3 |
| Hair Alignment | Good | Good | Good | Good | Control |
| Shape Recovery | Good | Good | Good | Good | Control |

High Molecular Weight Chitosan: 2% by weight
Fatty Acid: Isostearic Acid

TABLE 12

| | | | | | Comp. |
|---|---|---|---|---|---|
| | Ex. 24 | Ex. 25 | Ex. 26 | Ex. 27 | Ex. 4 |
| Hair Alignment | Good | Good | Good | Very | Control |
| | | | | Good | |
| Shape Recovery | Good | Good | Good | Good | Control |

High Molecular Weight Chitosan: 2% by weight
Fatty Acid: Linoleic Acid

TABLE 13

| | | | | | Comp. |
|---|---|---|---|---|---|
| | Ex. 28 | Ex. 29 | Ex. 30 | Ex. 31 | Ex. 5 |
| Hair Alignment | Good | Good | Good | Good | Control |
| Shape Recovery | Good | Good | Good | Good | Control |

High Molecular Weight Chitosan: 2% by weight
Fatty Acid: Erucic Acid

TABLE 14

| | | Comp. | Comp. | Comp. | Comp. | Comp. |
|---|---|---|---|---|---|---|
| | Ex. 1 | Ex. 6 | Ex. 7 | Ex. 8 | Ex. 9 | Ex. 10 |
| Hair | Very | Poor | Poor | Poor | Poor | Control |
| Alignment | Good | | | | | |
| Shape | Good | Poor | Poor | Poor | Poor | Control |
| Recovery | | | | | | |

High Molecular Weight Chitosan: 2% by weight
Low Molecular Weight Chitosan: 2% by weight
Fatty Acid: Oleic Acid

### SUMMARY

According to Tables 9 and 10, it can be found that the composition according to the present invention including a various crosslinker showed good or very good effects regarding hair alignment and shape recovery, as compared to the composition including no crosslinker.

According to Tables 11-13, it can be found that the composition according to the present invention including a various fatty acid showed good effects regarding hair alignment and shape recovery, as compared to the composition including no crosslinker.

According to Table 14, it can be found that, using low molecular weight chitosan with a molecular weight of less than 5,000 resulted in poor effects regarding hair alignment and shape recovery, as compared to the composition including no crosslinker. Using high molecular weight chitosan with a molecular weight of more than 20,000 resulted in very good effects regarding hair alignment and shape recovery, as compared to the composition using low molecular weight chitosan with a molecular weight of less than 5,000.

Moreover, the composition according to the present invention includes environmentally-friendly ingredient(s)

1-15. (canceled)

16. A composition for treating keratin fibers, comprising:
(a) one or more cationic polymers chosen from chitosans with a molecular weight of at least 20,000;
(b) one or more monovalent non-polymeric acids or salts thereof; and
(c) one or more non-polymeric acids having two or more pKa values or salts thereof.

17. The composition according to claim 16, wherein the total amount of (a) cationic polymer(s) ranges from 0.01% to 15% by weight, relative to the total weight of the composition.

18. The composition according to claim 16, wherein the (b) monovalent non-polymeric acid(s) are chosen from monovalent non-polymeric organic acids.

19. The composition according to claim 16, wherein the total amount of (b) monovalent non-polymeric acid(s) and salt(s) thereof ranges from 0.01% to 15% by weight, relative to the total weight of the composition.

20. The composition according to claim 16, wherein the (c) non-polymeric acid(s) having two or more pKa values are chosen from organic acids.

21. The composition according to claim 16, wherein the total amount of (c) non-polymeric acid(s) having two or more pKa values and salt(s) thereof ranges from 0.001% to 10% by weight, relative to the total weight of the composition.

22. The composition according to claim 16, wherein the composition further comprises (d) water.

23. The composition according to claim 16, wherein the composition further comprises (e) one or more oils.

24. The composition according to claim 23, wherein the (e) oil(s) are chosen from plant oils, synthetic ester oils, and mixtures thereof.

25. The composition according to claim 24, wherein the total amount of (e) oil(s) ranges from 1% to 50% by weight, relative to the total weight of the composition.

26. The composition according to claim 23, wherein the composition further comprises (f) one or more fatty acids.

27. The composition according to claim 26, wherein the (f) fatty acid(s) are chosen from C₄-C₂₆ saturated or unsaturated, linear or branched fatty acids.

28. The composition according to claim 26, wherein the (a) cationic polymer(s) are hydrophobized by the (f) fatty acid(s).

29. The composition according to claim 26, wherein the total amount of (f) fatty acid(s) ranges from 0.01% to 15% by weight, relative to the total weight of the composition.

30. A method for treating keratin fibers, comprising:
(i) applying to the keratin fibers a composition comprising:
(a) one or more cationic polymers chosen from chitosans with a molecular weight of at least 20,000;
(b) one or more monovalent non-polymeric acids or salts thereof; and
(c) one or more non-polymeric acids having two or more pKa values or salts thereof, and
(ii) drying the composition to form a cosmetic film on the keratin fibers.

31. The method according to claim 30, wherein the total amount of (a) cationic polymer(s) ranges from 0.01% to 15% by weight, relative to the total weight of the composition.

32. The method according to claim 30, wherein the total amount of (b) monovalent non-polymeric acid(s) and salt(s) thereof ranges from 0.01% to 15% by weight, relative to the total weight of the composition.

33. The method according to claim 30, wherein the total amount of (c) non-polymeric acid(s) having two or more pKa values and salt(s) thereof ranges from 0.001% to 10% by weight, relative to the total weight of the composition.

34. The method according to claim 30, wherein the composition comprises:
(a) one or more cationic polymers chosen from chitosans with a molecular weight of at least 20,000;
(b) one or more monovalent non-polymeric acids or salts thereof;
(c) water;
(d) optionally, one or more oils; and
(e) optionally, one or more fatty acids.

35. The method according to claim 30, wherein the (b) monovalent non-polymeric acid(s) are chosen from monovalent non-polymeric organic acids, and/or the (c) non-polymeric acid(s) having two or more pKa values are chosen from organic acids.
